(19) United States
(12) Patent Application Publication
(10) Pub. No.: US 2023/0404779 A1
(43) Pub. Date: Dec. 21, 2023

(54) Damper unit and orthopedic device

(71) Applicant: OTTO BOCK HEALTHCARE PRODUCTS GMBH, Vienna (AT)

(72) Inventor: Florian EDER, Vienna (AT)

(21) Appl. No.: 18/252,465

(22) PCT Filed: Nov. 10, 2021
(86) PCT No.: PCT/EP2021/081215

§ 371 Date: May 10, 2023

(30) Foreign Application Priority Data
Nov. 12, 2020 (DE) 10 2020 129 853.6

(51) Int. Cl. A61F 2/64; F16F 9/06; F16F 9/34

(52) U.S. Cl. CPC A61F 2/64; F16F 9/062; F16F 9/34; A61F 2002/5006

(57) ABSTRACT

The invention relates to a damper unit (40) comprising: a hydraulic cylinder (50) having a hydraulic piston (51) movably mounted therein, which hydraulic piston is coupled to a piston rod (70) and divides the hydraulic cylinder (50) into two hydraulic chambers (52, 53) which are fluidically interconnected via at least one hydraulic channel (54); and a pneumatic cylinder (60) having a pneumatic piston (61) movably mounted therein, which pneumatic piston is coupled to the piston rod (70) and divides the pneumatic cylinder (60) into two pneumatic chambers (62, 63) which are fluidically interconnected via at least one pneumatic channel (64); wherein the value of the volume change of the hydraulic chambers (52, 53) during a movement of the hydraulic piston (51) is different and the hydraulic chambers (52, 53) are fluidically coupled to an equalisation volume (80).

The invention relates to a damper unit having a hydraulic cylinder with a hydraulic piston which is mounted displaceably therein and which is coupled to a piston rod and divides the hydraulic cylinder into two hydraulic chambers which are fluidically interconnected via at least one hydraulic channel, and having a pneumatic cylinder with a pneumatic piston which is mounted displaceably therein and which is coupled to the piston rod and divides the pneumatic cylinder into two pneumatic chambers which are fluidically interconnected via at least one pneumatic channel. The invention likewise relates to an orthopedic device having an upper part and a lower part which are mounted pivotably on each other via a joint, and having a damper unit, as is described above. The orthopedic device is designed in particular as an orthosis, an exoskeleton or a prosthesis, in particular as an artificial knee joint, but is not limited to such applications.

In order, for example, to control a movement behavior of two components, relative movements between two components are influenced via damper devices. The damper device arranged in each case between the two components provides a resistance to the relative movement, said resistance being able to be adjusted. A frequent embodiment of the damper device provides a linear hydraulic damper which is arranged in a housing. A cylinder in which a piston which is arranged on a piston rod is moved is formed or arranged within the housing. The piston rod is coupled to a first component, and the housing or the cylinder to the second component. The cylinder is filled with a hydraulic fluid which is conveyed during a movement of the piston from the hydraulic chamber which is decreased in size into the enlarging hydraulic chamber. The conveying can be undertaken either through the piston or through one or more hydraulic channels within the housing. In the hydraulic channel or in the hydraulic channels there are throttle devices to increase or adjust the flow resistance. The throttle devices can be designed adjustably, for example in the form of actuating valves or switching valves, such that a variable flow resistance can be provided. Adjustable throttle devices are generally not arranged in a piston. A hydraulic damper device is known, for example, from EP 2 285 315 B1.

In addition to hydraulic damper devices, pneumatic damper devices are known which have an identical structural design to hydraulic damper devices.

DE 10 2016 118 999 A1 discloses an actuator damper unit for use in orthotic and prosthetic devices, in which a cylinder in which a first piston is movably mounted and is coupled to a piston rod is formed in a housing. At least one further piston is coupled to the first piston so as to form a further, volume-changeable fluid chamber, and the fluid chamber can be designed, for example, as a pneumatic spring.

U.S. Pat. No. 6,517,582 B1 relates to a prosthetic leg with a prosthetic knee joint and a hydraulic resistance device which is adjustable in respect of the hydraulic resistance via a sensor-controlled control device. In addition to the hydraulic resistance device, a pneumatic resistance device is provided. The control device can adjust the hydraulic and pneumatic resistance against a flexion of the knee.

It is the object of the present invention to provide a damper unit and an orthopedic device with which it is possible in a simplified way to provide resistance against a first movement and support during a reversal of the direction of movement.

According to the invention, this object is achieved by a damper unit and an orthopedic device having the features of the independent claims. Advantageous embodiments and developments of the invention are disclosed in the dependent claims, the description and the figures.

The damper unit having a hydraulic cylinder with a hydraulic piston which is mounted displaceably therein and which is coupled to a piston rod and divides the hydraulic cylinder into two hydraulic chambers which are fluidically interconnected via at least one hydraulic channel, and having a pneumatic cylinder with a pneumatic piston which is mounted displaceably therein and which is coupled to the piston rod and divides the pneumatic cylinder into two pneumatic chambers which are fluidically interconnected via at least one pneumatic channel, makes provision that the value of the volume change of the hydraulic chambers during a movement of the hydraulic piston differs and the hydraulic chambers are fluidically coupled to a compensating volume. By coupling of the hydraulic cylinder in the damper unit to a pneumatic cylinder, it is possible to supplement a hydraulically damped movement by a gas spring or a pneumatic spring. During the braking movement by the hydraulic cylinder, it is simultaneously possible, by compression in a pneumatic chamber, to pressurize the gas volume located therein or the volume located therein of a compressible liquid and, during a reversal of the movement in the opposite direction, to release the compression energy again in order thereby to bring about or to assist an oppositely directed movement of hydraulic cylinder and pneumatic cylinder and therefore also of the piston rod. The hydraulic damping is assisted by the braking phase during the charging of the pneumatic chamber and the stored kinetic energy assists the movement in the opposite direction during the relaxation of the compressed gas or of the compressed compressible fluid. In particular when the damper unit is used in an artificial knee joint, for example a prosthetic knee joint or orthotic knee joint, the resilient bending is permitted at the beginning of the stance phase, what is referred to as stance phase flexion, and assists the stance phase extension. Where compressible or compressed gases are referred to below, the statements also apply to compressible fluids.

A development of the invention makes provision that the value of the volume change of the pneumatic chambers during movement of the pneumatic piston is identical. This is achieved in particular in that both sides of the pneumatic piston have the same effective surface areas. By means of the effective piston surface areas of equal size and the identical values of the changing volumes in the two pneumatic chambers, it is possible to allow the pneumatics to become ineffective via the pneumatic channel such that the pneumatics do not provide any forces when a pneumatic channel is open. This means that the pneumatics can be filled with much higher pressures without developing an inadvertent extension assist effect, as a result of which high charging of the compressed pneumatic chamber can be achieved and thus high forces can be absorbed and applied.

In one embodiment, the pneumatic chamber is arranged on the piston rod, the piston rod projecting through the pneumatic cylinder. In this case, the piston rod does not have to be an integral component; there is likewise the option of two mutually opposite piston rod sections being arranged or fastened on opposite sides of the pneumatic piston. By means of the continuous piston rod with identical cross sections, same effective piston surface areas are provided in the pneumatic cylinder. In one variant, the pneumatic piston is fastened to the hydraulic cylinder, the hydraulic cylinder being arranged movably in the pneumatic cylinder. In this embodiment, the hydraulic piston is located within the hydraulic cylinder, and, by contrast, the pneumatic piston is arranged on the outer side of the hydraulic cylinder and is displaced together with the hydraulic cylinder within the pneumatic cylinder. This leads to a reduction in the overall height because of the internested design. It is accepted here that, because of the different piston rod diameters, different pneumatic forces arise when a pneumatic channel is open. However, for certain applications, this may be entirely desirable.

In one development, at least one switchable or adjustable hydraulic valve is arranged in the hydraulic channel, which hydraulic valve or hydraulic valves make it possible to adjust the hydraulic resistance. In particular the hydraulic valve is adjusted or switched on the basis of sensor data which are transmitted to a control device which, in turn, activates and/or deactivates an actuator for opening, closing or adjusting the hydraulic valve. In particular, two actuating valves are arranged in the hydraulic channel or in the hydraulic channels, one for adjusting a flexion resistance and the other for adjusting the extension resistance. Two check valves arranged in parallel and a connecting channel to the compensating volume can furthermore be provided in order to be able to provide a variable hydraulic resistance which is adapted to the respective situation.

In one embodiment, the compensating volume is closed and has an air bubble or gas bubble which is compressed during the filling of the compensating volume. As a result, the hydraulic fluid is pre-pressurized by the compressed gas and can be used by corresponding valve switching to drive or assist the respectively desired movement of the piston rod. In addition to pneumatic spring pre-pressurization of the hydraulic fluid, this can also be undertaken mechanically, for example via a spring-loaded piston within the compensating volume.

In one embodiment of the invention, at least one switchable or adjustable pneumatic valve is arranged in the pneumatic channel and can be used to switch the pneumatics to be ineffective. When a pneumatic valve is open and with identical effective surface areas of the pneumatic piston, it is possible to displace the gas located in the pneumatic system between the two chambers without force. The pneumatics will therefore not provide a substantial portion of a resistance against movement of the piston rod. It is therefore possible to switch the pneumatics on or off irrespective of the filling pressure in the pneumatic system. If the pneumatic valve is closed in different piston positions, it is possible via this for the zero point of the then resulting pneumatic spring to be displaced. The use of the spring in different positions of the piston rod is therefore made possible. When the damper device is used in a lower extremity, the use of a pneumatic spring in different gait phases or movement phases can therefore be made possible. In addition to the use in the stance phase flexion, a pneumatic spring can also be used, for example, for assisting the extension movement at the end of the swing phase flexion. A displacement of the zero point of the spring with a spring behavior remaining virtually the same is therefore easily possible because of the effective piston surface areas of identical size. The displacement of a zero point of the pneumatic spring can therefore be simply adjusted by opening and closing of a single pneumatic valve.

In one development, provision is made that in the pneumatic channel there are arranged two pneumatic valves with respect to which two mutually oppositely switched check valves are arranged in a parallel channel, the parallel channel being connected to the pneumatic channel via a connecting channel between the pneumatic valves and the check valves. By means of such circuitry of the pneumatic chambers to two actuating valves or switching valves and two check valves, it is possible to prepare the pneumatic part of the damper device before an anticipated switching and to open or to close the respective valves. The preparatory opening and/or closing of the pneumatic valves means that it is no longer necessary to switch them directly when a certain position of the piston or of the piston rod is reached, as a result of which a greater time window for the actuation of the respective pneumatic valve is produced. For example, a pneumatic flexion valve can already be closed when an extension movement is still being carried out, and it can be anticipated that flexion assistance will be undertaken at the end of the extension movement.

A fluidic connection which is closed or is closeable via a filling valve can be formed between the connecting channel and the surrounding atmosphere. For example, the filling pressure in the pneumatic system can be adapted via the filling valve.

The orthopedic device having an upper part and a lower part which are mounted pivotably on each other via a joint, and having a damper unit, as has been described above, makes provision that said damper unit is arranged between the upper part and the lower part and provides resistance to pivoting of the upper part relative to the lower part. In addition to the provision of the resistance because of the hydraulic flow resistance and optionally the pneumatic flow resistance, it is possible to provide assistance of a movement because of the compression which is present of a pneumatic chamber.

One development makes provision that the at least one pneumatic valve is assigned an actuator which is coupled to a control device, which is coupled to sensors or to an operator control device and adjusts the at least one pneumatic valve on the basis of the sensor values and/or commands via the operator control device. The pneumatic valve can also be actuated via a purely mechanical control device without an actuator having an energy store having to be present. The pneumatic valve can close via a mechanism, e.g. in the event of an axial load on the lower leg, and can open, e.g., when the axial load ceases.

One development makes provision for the orthopedic device to be designed as an orthosis or prosthesis of a lower extremity, in particular as an artificial knee joint. It is possible via the damper unit, in an embodiment as an artificial knee joint, to achieve a lower and therefore physiologically more natural stance phase flexion since a stance phase extension can be assisted by the pneumatic spring. By the compression and decompression in the respective pneumatic chamber, energy can be stored in the damper unit and dispensed again, and therefore the damper unit serves simultaneously as an actuator or a movement-assisting device of the orthopedic device. The spring rigidity of the pneumatic spring can simply be adapted by the pressure within the pneumatic system. For example, it can be adapted to the weight of the user or to the personal preferences of the user. No additional outlay on control for the pneumatic component during the sprung stance phase is necessary since valves are not switched over during the changing of the direction of movement.

Exemplary embodiments will be explained in more detail below with reference to the figures, in which:

FIG. 1—shows a perspective illustration of an orthopedic device with a damper unit;

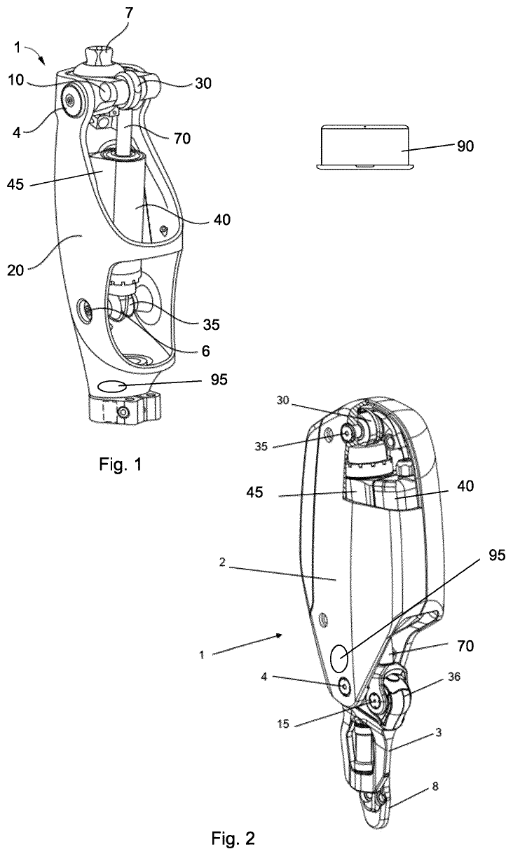

FIG. 2—shows a variant of FIG. 1 in the form of an orthosis;

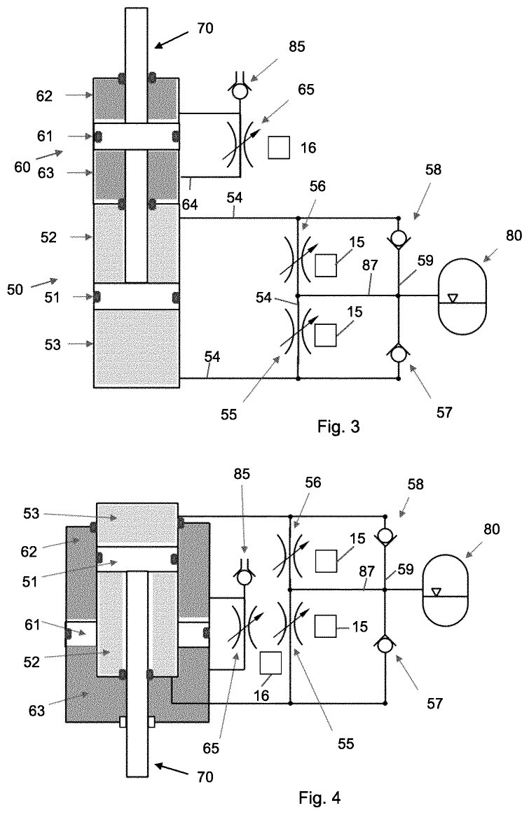

FIG. 3—shows a circuit diagram of a first variant of the damper unit;

FIG. 4—shows a variant with a movable hydraulic cylinder in a pneumatic cylinder; and

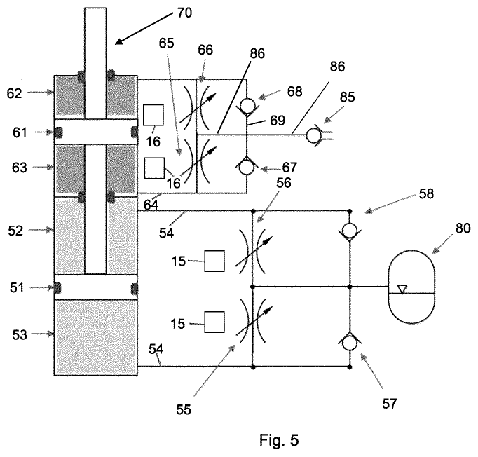

FIG. 5—shows a variant of FIG. 3 with two pneumatic actuating valves.

FIG. 1 shows, in a perspective illustration, an orthopedic joint device 1 in the form of a prosthetic knee joint. The orthopedic joint device 1 has an upper part 10 and a lower part 20 which are mounted pivotably on each other about a joint axis 4. The lower part 20 is designed as a three-dimensional hollow body which has an actuator or a damper unit 40 with a piston rod 70. The upper part 10 has, at its proximal end, a device 7 for fastening a proximal component, for example an upper leg tube or an upper leg socket. In the exemplary embodiment illustrated, the fastening device 7 is designed as a pyramid adapter; other embodiments are also possible. Furthermore, a head as bearing point or bearing seat 30, which is arranged on or fastened to a proximal end of the piston rod 70, is mounted on the upper part 10 so as to be pivotable about an axis or a bolt. Furthermore, the distal end of the damper unit 40 is mounted on a distal bearing point or a distal bearing seat 35 so as to be pivotable about an axis 6. The damper unit 40 can be secured releasably both to the distal bearing point 35 and to the head or the proximal bearing seat 30, in particular via a screw connection or a snap connection.

In addition to an embodiment of the orthopedic joint device 1 as a prosthesis knee joint, the latter can also be designed as an orthotic knee joint or other joint device, as is shown in FIG. 2. FIG. 2 shows, in a perspective illustration, an alternative embodiment of the orthopedic device 1, namely as an orthosis component. An upper part 10 in the form of a housing for accommodating the damper unit 40 is arranged pivotably about a joint axis 4 on a lower part 20, which can be fastened to an orthosis rail, for example via a plate 8 articulated thereto. The upper part 10 is also fixed to an orthosis rail. The two orthosis rails (not shown) are then fastened to a limb, for example via shells, straps, buckles or other devices for fixing the orthosis on the limb. The orthopedic device 1 therefore forms a joint between the two orthosis rails.

The damper unit 40, as illustrated in FIGS. 1 and 2, has a hydraulic damper component and a pneumatic damper component, the design of which will be explained further below.

Both in the embodiment as a prosthesis and in the orthosis, the damper unit 40 is coupled to a control device 45 in which the required hardware and software components for processing sensor data and for activating and deactivating actuators are arranged. An energy supply or energy store can also be present in the control device 45. Interfaces for transmitting data and/or transmitting energy are also assigned to the control device 45. In the two embodiments according to FIGS. 1 and 2 there are sensors 95, illustrated schematically, which record data, for example spatial position data, loading data, angle data, temperatures or other parameters or changes thereof, on the basis of which corresponding signals in the control device 45 for activating or deactivating actuators to adjust actuating valves are activated or deactivated. Alternatively or additionally, these valves can be adjusted or set via an operator control device 90, which is illustrated by way of example as a computer. The communication with the control device 45 is undertaken wirelessly or via a cable connection. On the damper unit 40 there can also be ports for a compressor or a pump in order to be able to adapt the pressure level in the pneumatic component to what is necessary. Alternatively, a pump for filling and charging the damper unit 40 can be integrated in the control device 45.

FIG. 3 shows a schematic illustration of a first variant of the damper unit 40 in an uninstalled state. A hydraulic cylinder 50 has a hydraulic piston 51 which is mounted displaceably therein and has a seal on its circumference, and therefore the hydraulic cylinder 50 is divided into two hydraulic chambers 52 and 53. Fluid is exchanged between the hydraulic chambers 52, 53 via a hydraulic channel 54, in which two actuating valves 55, 56 are arranged. The actuating valves 55, 56 are assigned actuators 15 in order to change the flow cross section and therefore the hydraulic resistance. The actuators 15 are coupled to the control device 45, not illustrated. Parallel to the hydraulic channel 54 with the two actuating valves 55, 56, a parallel channel 59 is formed in which two check valves 57, 58 are arranged oriented oppositely to each other. A hydraulic connecting channel 87, which leads into a compensating volume 80, is formed between the two actuating valves 55, 56 and between the two check valves 57, 58.

A pneumatic cylinder 50 through which the piston rod 70 passes is arranged above the hydraulic cylinder 50. A pneumatic cylinder 61 is arranged on the piston rod 70 and, corresponding to the hydraulic cylinder 51, has a peripheral seal and divides the pneumatic cylinder 60 into two pneumatic chambers 62, 63. Between the two pneumatic chambers 62, 63 there is formed a pneumatic channel 64 in which a pneumatic valve 65 as an actuating valve is arranged which can be adjusted via an actuator 16, which is coupled to the control device 45. A filling valve 85 is connected to the pneumatic channel 64 and makes it possible to adjust the filling pressure within the pneumatic chambers 62, 63. Pressurized gas can also be let out via the filling valve 85.

The piston rod 70 can be formed integrally and can pass through the pneumatic cylinder 60. Alternatively, the piston rod 70 is formed in two parts and extends through the pneumatic piston 60 on either side of the pneumatic cylinder 61. The transition of the connecting section of the piston rod 70 between the pneumatic piston 61 and the hydraulic piston 51 is sealed so that no hydraulic fluid enters the pneumatic cylinder 60 and, conversely, no gas enters the hydraulic cylinder 50. The piston rod 70 does not pass through the hydraulic cylinder 50, and the hydraulic piston 51 forms the end of the piston rod 70 such that the piston rod 70 projects out of the hydraulic cylinder 50 only on one side. This saves on construction height of the damper unit 40 since the hydraulic and pneumatic cylinders 50, 60 which are arranged one above the other do not have to be extended by a piston rod 70 projecting through them without connection to a component of the orthopedic device. The compensating volume 80 is provided because of the different volumes of the hydraulic chambers 52, 53 owing to the piston rod 70 being located only in one hydraulic chamber 52. Furthermore, the compensating volume 80 serves as a storage volume for, for example, evaporating hydraulic fluid and can also be used as an energy store. For this purpose, a gas bubble or a spring pressing against a piston is arranged in the compensating volume 80, and therefore the spring or gas bubble is compressed each time the piston rod is retracted, and is expanded each time it is extended, the expansion assisting the extension of the piston rod.

FIG. 4 shows a variant of the design of the damper unit 40. Identical reference numbers denote identical components. The basic design of the hydraulic circuitry corresponds to that of FIG. 3. In contrast to the embodiment of the hydraulic cylinder 50 and the pneumatic cylinder 60 arranged one above another or one behind another, in FIG. 4 the hydraulic cylinder 50 is arranged movably within the pneumatic cylinder 60. The pneumatic piston 61, which is moved within the pneumatic cylinder 60 together with the hydraulic cylinder 50, is arranged on the outer side of the hydraulic cylinder 50. The hydraulic cylinder 50 projects upward out of the pneumatic cylinder 60 and is sealed at the passage point. Similarly, the piston rod 70 projects out of the hydraulic cylinder 50, the sealing also being shown. The pneumatic cylinder 60 is fixedly connected to the piston rod 70. The upper part of the orthopedic device is arranged either on the piston rod 70 or the hydraulic cylinder 50, the lower part on the respective other component. A compensating volume 80 is also provided here; unlike in the embodiment according to FIG. 3, there is a different volume of the pneumatic chambers 62, 63 in this embodiment.

FIG. 5 illustrates a further variant which substantially corresponds to the design of FIG. 3. The pneumatic circuitry corresponds to the hydraulic circuitry, that is to say there are two pneumatic valves 65, 66 which can be adjusted via actuators 16. Two check valves 67, 68 are arranged oppositely oriented in a parallel channel 69, and a connecting channel 86 produces a connection to the filling valve 85. The connecting channel 86 is arranged between the actuating valves or pneumatic valves 65, 66 and the check valves 67, 68.

By means of the embodiment of the damper unit 40 with a pneumatic component and a hydraulic component and by means of the embodiment of the hydraulic component with a non-continuous piston rod, the required compensating volume 80 can be used to compensate for volume changes occurring during operation because of thermal expansion or oil evaporation. As a result, in addition to the saving on construction space by omitting a continuous piston rod 70, the operational reliability is increased. Furthermore, the compensating volume 80 can be pressurized, either with pneumatic pressure or mechanical spring pressure, so that an extension assist effect can be achieved by the hydraulic component of the damper unit 40 via the compensating volume 80.

The pneumatic component of the damper unit 40 has effective piston surface areas of identical size because of the continuous piston rod 70 or the piston rod 70 formed at least on either side of the pneumatic piston 61, and therefore, when the pneumatic piston 61 is moved, a volume change of identical size is thereby produced in the two pneumatic chambers 62, 63 according to the embodiment of FIGS. 3 and 5. The volume change is identical in terms of value, and the reduction in volume of one chamber leads to a corresponding increase in volume of the other chamber. As a result, it is possible to switch the pneumatics to be ineffective solely by opening a single pneumatic valve 65. When a pneumatic valve 65 is open, the displacement of the air volume or gas volume within the pneumatic chambers 62, 63 requires only a negligible force, if any at all, and therefore no damping effect takes place. The pneumatic valve 65 can be closed in any desired position of the pneumatic cylinder 61, and therefore a pneumatic spring can thereby be formed by the pneumatic cylinder 60. The zero point of said spring can be selected as desired. Owing to the displacement volumes of identical size in the pneumatic chambers 62, 63, the pneumatic cylinder can also be filled with very high pressures, and therefore, for example, when used in artificial knee joints, a controllable deflection effect and restoring assistance can be achieved.

Furthermore, the use of a pneumatic spring in different movement situations or adjustment situations can be achieved by adjusting the valve or the valves. In the case of an artificial knee joint, the spring effect can be brought about by closing of the pneumatic valve at the designated position of the pneumatic piston 61 at a relatively small flexion of a flexion angle of 4° to 8° in order to assist a stance phase extension. Similarly, it is possible to bring about a swing phase reversal at a flexion angle or 30° or more in the swing phase by the pneumatic valve being correspondingly adjusted.

If an extension effect of the pneumatic piston can be accepted when a pneumatic valve is open, the embodiment according to FIG. 4 is advantageous because of the compact design and the shorter overall height of the damper unit. A comparatively high extension assist effect can be anticipated because of the different piston rod diameters.

In the case of circuitry of the pneumatic component as shown in FIG. 5, an anticipating positioning of the pneumatic valves 65, 66 can be achieved. The actuating valves 65, 66 do not have to be switched instantaneously, but rather can be brought beforehand into the respectively desired position. For example, during the terminal stance phase when used in an artificial knee joint, the flexion actuating valve 65 can be opened. In the terminal stance phase, the pneumatic piston is pressed here into the upper working pneumatic chamber 62 while the pneumatic extension actuating valve 66 remains closed. The pneumatic extension spring assists the introduction of the swing phase, that is to say that the pneumatic piston 61 is pressed downward. As soon as the pneumatic spring is relaxed, i.e. the same pressure prevails in the two pneumatic chambers 62, 63 and the pneumatic piston 61 is lowered further, the gas flows via the flexion actuating valve 65 and the flexion check valve 68 back into the pneumatic chamber 62.

1. A damper unit, comprising:
a hydraulic cylinder;
a hydraulic piston mounted displaceably within the hydraulic cylinder; therein and which is coupled to
a piston rod which divides the hydraulic cylinder into two hydraulic chambers which are fluidically interconnected via at least one hydraulic channel, wherein the hydraulic piston is coupled to the piston rod;
a pneumatic cylinder;
a pneumatic piston mounted displaceably within the pneumatic cylinder, wherein the pneumatic piston is coupled to the piston rod, wherein the pneumatic piston divides the pneumatic cylinder into two pneumatic chambers which are fluidically interconnected via at least one pneumatic channel,
wherein a value of a volume change of the two hydraulic chambers during a movement of the hydraulic piston differs, and
wherein the two hydraulic chambers are fluidically coupled to a compensating volume.

2. The damper unit as claimed in claim 1, wherein the value of the volume change of the two pneumatic chambers during a movement of the pneumatic piston is identical.

3. The damper unit as claimed in claim 1, wherein the pneumatic piston is arranged on the piston rod and the piston rod projects through the pneumatic cylinder.

4. The damper unit as claimed in claim 1, wherein the pneumatic piston is fastened to the hydraulic cylinder and the hydraulic cylinder is arranged movably in the pneumatic cylinder.

5. The damper unit as claimed in claim 1, further comprising at least one switchable or adjustable hydraulic valve is arranged in the at least one hydraulic channel.

6. The damper unit as claimed in claim 1, further comprising at least one switchable or adjustable pneumatic valve is arranged in the at least one pneumatic channel.

7. The damper unit as claimed in claim 1, wherein in the at least one pneumatic channel there are arranged two pneumatic valves with respect to which two mutually oppositely switched check valves are arranged in a parallel channel, wherein the parallel channel is connected to the at least one pneumatic channel via a connecting channel between the two pneumatic valves and the two mutually oppositely switched check valves.

8. The damper unit as claimed in claim 7, wherein the connecting channel provides a fluidic connection to a surrounding atmosphere and is closed via a filling valve.

9. An orthopedic device, comprising:
an upper part;
a lower part, wherein the upper part and the lower part are mounted pivotably on each other via a joint; and
a damper unit as claimed in claim 1, wherein the damper unit is arranged between the upper part and the lower part and provides resistance to pivoting of the upper part relative to the lower part.

10. The orthopedic device as claimed in claim 9, wherein the at least one pneumatic valve is assigned an actuator which is coupled to a control device, wherein the control device is coupled to sensors or to an operator control device and adjusts the at least one pneumatic valve on a basis of sensor values and/or commands via the operator control device.
